(19) United States
(12) Patent Application Publication
(10) Pub. No.: US 2025/0073081 A1
(43) Pub. Date: Mar. 6, 2025

(54) LASER EYE SURGERY SYSTEM EMPLOYING DUAL-CHANNEL VIDEO IMAGING SYSTEM FOR REAL-TIME PROCEDURE VISUALIZATION AND RELATED IMAGING METHODS

(71) Applicant: AMO Development, LLC, Irvine, CA (US)

(72) Inventors: Hong Fu, Pleasanton, CA (US); Mohammad Saidur Rahaman, San Jose, CA (US)

(21) Appl. No.: 18/817,116

(22) Filed: Aug. 27, 2024

Related U.S. Application Data
(60) Provisional application No. 63/580,979, filed on Sep. 6, 2023.

(51) Int. Cl. A61F 9/008; A61B 3/00; A61B 34/20

(52) U.S. Cl. CPC A61F 9/0084; A61B 3/0058; A61B 2034/2057

(57) ABSTRACT

In a femtosecond laser eye surgery system where beam delivery is accomplished with a moving objective, a dual-channel imaging system allows real-time procedure visualization before and during incision. The first (docking) imaging channel covers a full field of view (FoV) of the eye, e.g., 13 mm; the second (cutting) imaging channel is through the objective and moves with it, and covers a smaller FoV, e.g., 2 mm. During eye docking and undocking, the objective is moved to a parking position out of the visual field of the docking imaging channel, and the latter operates to provide process visualization. During incision, a composite eye image is displayed, composed of a stationary image captured by the docking imaging channel before treatment began overlayed with live cutting images captured by the cutting imaging channel. The live cutting images are compared to the stationary image in real time to detect eye movement.

### CROSS-REFERENCE TO RELATED APPLICATIONS

This application claims priority to U.S. Provisional Application No. 63/580,979, filed Sep. 6, 2023, the entire contents of which are hereby incorporated by reference in its entirety.

### BACKGROUND OF THE INVENTION

This invention relates to laser eye surgery systems and methods, and in particular, it relates to a laser eye surgery systems employing dual-channel video imaging for real-time procedure visualization, and related methods.

Ophthalmic laser systems, such as femtosecond laser systems, are used in various eye surgeries, such as corneal refractive surgeries which make incisions in the cornea, to form corneal flaps, corneal lenticules, etc., or otherwise treat the eye. There are generally two types of laser beam delivery optics that are used in femtosecond lasers for eye surgery. One type, illustrated in FIG. 7, is a beam delivery optics which uses a large field of view (FoV, e.g., about 10 mm across) focusing objective. As shown in FIG. 7, in this system, the objective is stationary in the XY plane (the plane perpendicular to the optical axis of the beam delivery optics) during incision. The light reflected and scattered from the eye is collected by the objective of the laser beam delivery optics, and directed by a beam splitter into the imaging camera which is a full FoV camera. An advantage of this system is that a coaxial large FoV imaging system can be designed for real-time procedure visualization. A challenge for this system is that it is difficult to achieve small focus at the periphery of the surgical field. Also, such systems can become increasingly more expensive for high numerical apertures (NA), such as NA≥0.4.

Another type of beam delivery optics uses a small FoV (e.g., about 2 mm across). In such a system, the focusing objective is moved during cutting to cover the surgical field which is typically 10 mm across. Advantages of this system include high focusing quality and low cost. However, a coaxial full FoV image becomes impossible, because the focusing objective is moving and blocking the visualization light path. Some existing femtosecond lasers using this design have no real-time imaging capabilities during cutting.

### SUMMARY OF THE INVENTION

The present invention is directed to a laser eye surgery systems employing dual-channel video imaging. An object of the present invention is to allow real-time procedure visualization during incision or other treatment for a femtosecond laser system that uses a moving objective to scan the laser beam on or in the eye.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a laser eye surgery system for treating a patient's eye, which includes: a laser device configured to generate a pulsed laser beam; an objective configured to focus the pulsed laser beam to a laser focus spot in the eye; a movable stage, wherein the objective is mounted on the movable stage and the movable stage is configured to move the objective in two lateral directions perpendicular to an optical axis of the objective to scan the laser focus spot in the two lateral directions; a first imaging channel including a first camera and a first set of optics associated with the first camera, wherein the first set of optics is configured to direct a reflected light from the eye into the first camera, and wherein the first imaging channel is configured to generate images of a first field of view (FoV) of the eye; and a second imaging channel including a second camera and a second set of optics associated with the second camera, wherein the second set of optics is mounted on the objective and moves with the objective, wherein the second imaging channel is configured to generate images of a second FoV of the eye which is smaller than the first FoV; wherein the movable stage is further configured to move the objective and the second set of optics between an operation position at which the laser beam passing through the objective is delivered through a beam exit of the laser surgery system to the eye and the reflected light from the eye is collected by the objective to enter the second set of optics, and a parking position at which the objective and the second set of optics are out of a light path between the beam exit and the first imaging channel.

In some embodiments, the laser eye surgery system further includes: a display device; and a controller operatively coupled to the laser device, the movable stage, the first imaging channel, the second imaging channel, and the display device, wherein the controller is configured to execute a control program to: operate the laser device and the movable stage to scan the laser focus spot in the eye to perform a laser treatment; operate the first imaging channel to generate a first image of the eye before performing the laser treatment, the first image covering the first FoV; and while performing the laser treatment: operate the second imaging channel to generate a plurality of second images of the eye, the second images covering the second FoV which moves across the eye; generate a composite image of the eye, including the first image as a stationary image and the plurality of second images overlayed on the first image to replace corresponding portions of the first image, wherein each overlayed second image is located at a corresponding location defined by the second FoV relative to the first FoV and has a same object-to-image ratio as the first image, the composite image further including an indication of a current boundary of the second FoV; and display the composite image on the display device.

In some embodiments, the laser eye surgery system further includes a controller operatively coupled to the laser device, the movable stage, the first imaging channel, and the second imaging channel, wherein the controller is configured to execute a control program to: operate the laser device and the movable stage to scan the laser focus spot in the eye to perform a laser treatment; operate the first imaging channel to generate a first image of the eye before performing the laser treatment, the first image covering the first FoV; and while performing the laser treatment: operate the second imaging channel to generate a plurality of second images of the eye, the second images covering the second FoV which moves across the eye; and compare the plurality of second images to the first image in real time to detect any eye movement, including, for each second image, comparing a portion of the second image which has not been scanned by the laser focus spot to a corresponding portion of the first image to detect any eye movement.

In another aspect, the present invention provides a method implemented in a laser eye surgery system to treat a patient's eye, the system comprising a laser device configured to generate a pulsed laser beam, an objective configured to focus the pulsed laser beam to a laser focus spot in the eye, a movable stage configured to move the objective in two lateral directions perpendicular to an optical axis of the objective, a first imaging channel configured to generate images of a first field of view (FoV) of the eye, a second imaging channel configured to generate images of a second FoV of the eye which is smaller than the first FoV, and a display device, the method including: moving the objective to a parking position which is out of a light paths of a visual field of the first imaging channel; while the eye is being docked to the laser system, continuously capturing first images of the eye by the first imaging channel and displaying the first images on the display device; after the eye is docked to the laser system, generating a first stationary image of the eye by the first imaging channel, the first image covering the first FoV; moving the objective to an operation position at which the laser beam passing through the objective is delivered to the eye; scanning the objective to deliver a laser focus spot in the eye to perform a laser treatment; while performing the laser treatment: collecting and directing reflected light from the eye through the objective to the second imaging channel; continuously capturing a plurality of second images of the eye by the second imaging channel, the plurality of second images covering the second FoV which moves across the eye; generating a composite image of the eye, including the first stationary image as a stationary image and the plurality of second images overlayed on the first stationary image to replace corresponding portions of the first stationary image, wherein each overlayed second image is located at a corresponding location defined by the second FoV relative to the first FoV and has a same object-to-image ratio as the first image, the composite image further including an indication of a current boundary of the second FoV; displaying the composite image on the display device; comparing the plurality of second images to the first stationary image in real time to detect any eye movement, including, for each second image, comparing a portion of the second image which has not been scanned by the laser focus spot to a corresponding portion of the first stationary image to detect any eye movement; and generating an alarm signal or automatically pausing laser beam delivery when detecting an eye movement greater than a predefined threshold; and after performing the laser treatment, undocking the eye from the laser system.

In some embodiments, the first FoV has a diameter of 10-13 mm and the second FoV has a diameter of 1 to 3 mm.

It is to be understood that both the foregoing general description and the following detailed description are exemplary and explanatory and are intended to provide further explanation of the invention as claimed.

### BRIEF DESCRIPTION OF DRAWINGS

FIG. 1 illustrates a laser eye surgery system with dual channel imaging according to an embodiment of the present invention.

FIGS. 2 and 3 illustrate structures of an XY moving stage and a laser objective in the laser eye surgery system of FIG. 1.

FIG. 4 illustrates a composite image displayed during laser incision in the laser eye surgery system of FIG. 1.

FIG. 5 schematically illustrates examples of a cutting FoV during laser incision in the laser eye surgery system of FIG. 1.

FIG. 6 schematically illustrates a method implemented in the laser eye surgery system according to an embodiment of the present invention.

FIG. 7 illustrates a conventional laser eye surgery system.

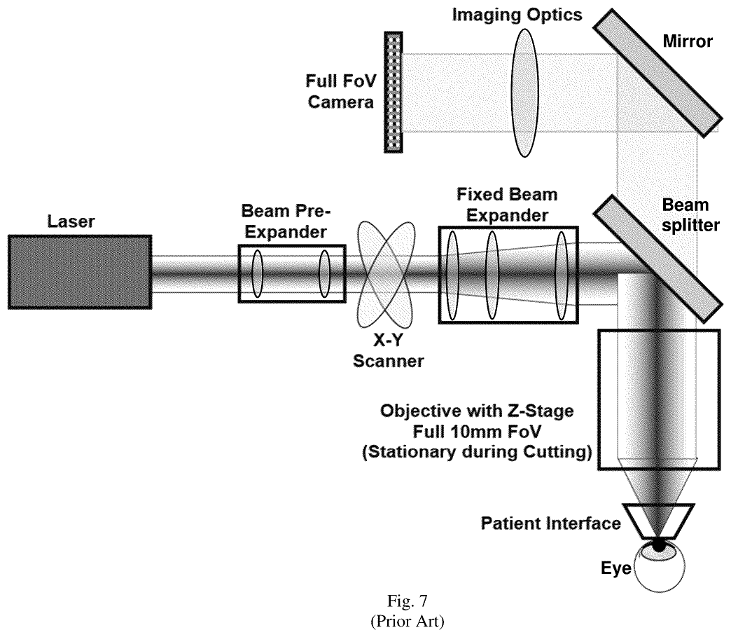

### DETAILED DESCRIPTION OF THE INVENTION

Embodiments of the present invention provide dual-channel imaging in a laser eye surgery system where beam delivery is accomplished with a moving focus objective. The dual channel imaging system includes the following sub-system components.

The first imaging channel covers a full FoV, for example, 10-13 mm diameter FoV, and preferably, 12-13 mm diameter FoV. It is used to visualize a patient's eye in real time for docking (a process of coupling the eye to the laser system using a patient interface device before performing laser treatment) and undocking (a process of un-coupling the eye from the patient interface device after performing laser treatment). For convenience, this imaging channel is referred to as the docking imaging channel. When using the docking imaging channel, the objective for the laser beam delivery (referred to as the cutting objective) is moved to a parking position which is out of the light paths of the visual field of the docking imaging channel.

The second imaging channel covers a small FoV (same as the FoV of the cutting objective), for example, 1 to 3 mm diameter, and preferably, 2 mm diameter, where the FoV moves with the cutting objective during laser treatment (incision, etc.). For convenience, this imaging channel is referred to as the cutting imaging channel. Using an image processing method described in more detail later, photodisruption bubble patterns in the eye can be observed and any eye movements can be monitored, which achieve two critical goals for real-time procedure visualization.

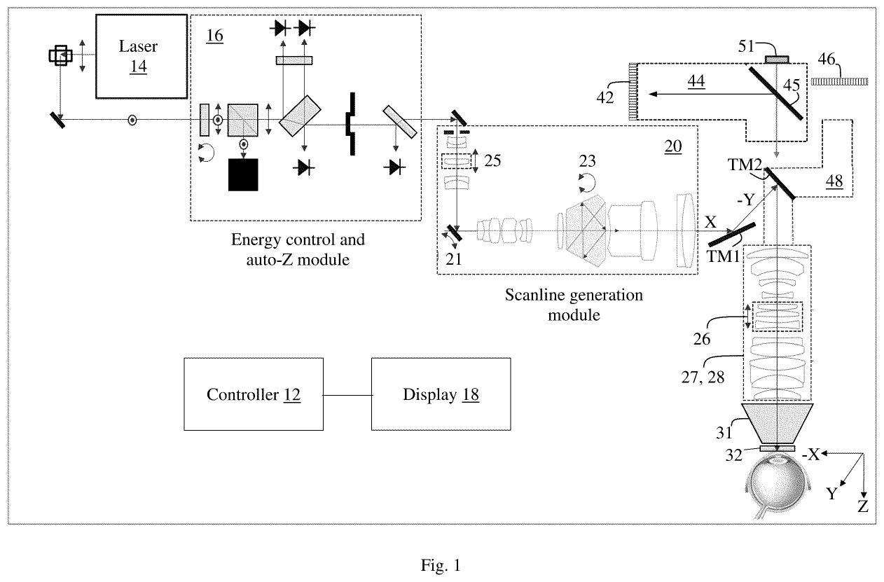

FIG. 1 illustrates a laser eye surgery system with dual channel imaging according to embodiments of the present invention. The system includes, inter alia, a laser device 14 for generating a pulsed laser beam, an energy control module 16 for varying the pulse energy of the pulsed laser beam, a scanline generation module (Keplerian telescope) 20 for generating a fast scanline of the pulsed laser beam, and a scanline movement control module 28 for moving the laser scanline and delivering it to the eye.

The laser device 14 may include a femtosecond laser, e.g. a femtosecond oscillator or a fiber oscillator-based low energy laser, which is capable of providing pulsed laser beams for optical procedures, such as localized photodisruption (e.g., laser induced optical breakdown). An exemplary set of laser parameters for such a laser include pulse energy in the 40-100 nJ range, pulse repetition rates in the 2-40 MHz range (preferably 10 MHz), pulse duration in the 100-200 fs range, and a wavelength range of 1015 nm to 1100 nm (preferably 1030 nm). Other suitable laser devices may be used, and the invention is not limited to particular laser devices or related parameters.

The energy control and auto-Z module 16 may include appropriate components to control the laser pulse energy, including attenuators and a shutter, as well as laser pulse energy monitoring components. It may also include an auto-Z module which employs a confocal or non-confocal imaging system to provide a depth reference.

The scanline generation module 20 includes a fast-Z scanner 25, a high frequency resonant scanner 21, and a scanline rotator 23. The fast-Z scanner 25, which includes a lens movable in the Z direction (i.e. direction of the optical axis of the beam delivery optics and laser beam propagation), scans the laser focus spot in the Z direction to control the treatment depth in the eye. The resonant scanner 21 scans the laser beam in the XY plane (the plane perpendicular to the Z direction, X and Y being two lateral direction perpendicular to the Z direction) back and forth to produce a laser scanline (formed of a line of laser focus spots) of 0.2-1.2 mm in length. The frequency of the resonant scanner is between 0.5 kHz and 20 kHz, preferably between 7 kHz and 9 kHz, for example, about 8 kHz. The scanline rotator 23, which may employ a prism (e.g., a Dove or Pechan prism or the like), rotates the laser scanline around the Z axis to place it in any direction on the XY plane. Note that the scanline generation module 20 is optional; the dual-channel imaging system may be implemented in a laser eye surgery system that does not employ a fast scanline.

The scanline movement control module 28 includes an objective lens 27 with Z-scanning capability, carried on a moveable XY-scanner (movable XY-stage), for delivering the laser beam focus spot to the eye. The movable XY-stage (also denoted by reference symbol 28) moves the objective lens 27 in the XY directions to achieve scanning of the laser scanline in the X and Y directions. The objective lens 27 with Z-scanning capability is referred to as slow-Z scanner because its Z-direction movement is slower than that of the fast-Z scanner 25. The Z-scanning of the objective lens 27 is used to set the depth baseline of the scan pattern of the laser focus spot in the eye.

In one example, the numerical aperture (NA) of the laser delivery optics is approximately 0.6; the laser focus spot diameter (full-width half-maximum) produced by the system is approximately 1.0 μm; and the laser focus spot separation as delivered to the eye is approximately 1 μm, which can achieve continuous tissue-bridge-free dissection. Those skilled in the art will appreciate that other laser and optical system parameters may be used.

The laser system also includes a controller 12, such as a computer or processor having a memory and operable to execute suitable control software stored in the memory. The controller 12 is coupled to the laser device 14, the energy control module 16, the scanline generation module 20, and the scanline movement control module 28, and is operable to control these components to scan the pulsed laser beam on or in the eye according to a scan pattern. The controller 12 is also coupled to the docking and cutting imaging channels and performs image processing as described in more detail later. The laser system further includes a display device 18, such as a touch panel display or other suitable display devices, coupled to the controller 12 to display images captured by the docking and cutting imaging channels and other information, as described in more detail later.

When used to perform an ophthalmic procedure, the laser system also includes a disposable patient interface device 32, which includes a cone shaped shell for coupling to a cone shaped component (cone optics) 31 of the laser system, a flexible suction ring for engaging with the patient's eye, and a contact lens which may contact the surface of the eye.

The docking imaging channel includes a docking imaging camera 42, which may be a video camera operating in visible and/or infrared range, along with appropriate docking imaging optics 44 (e.g., focusing lenses, reflecting mirrors, etc.). The docking imaging camera 42 and associated optics 44 are stationary relative to the mechanical frame of the laser system, and their optical axis is aligned with the laser beam exit of the laser system (i.e. the cone optics 31, which is fixedly mounted on the laser system frame). The docking imaging optics may include a beam splitter 45, which is also configured to couple a fixation light, generated by a fixation light source 51 (e.g. a light emitting diode emitting a red light with a full-view angle of approximately 1 degree), into the eye for eye fixation purposes. As mentioned earlier, when the docking imaging channel is operated during docking and undocking, the cutting objective 27 is moved to a parking position which is out of the light paths of the visual field of the docking imaging channel, so the light reflected from the eye reaches the docking imaging optics (e.g., the beam splitter 45) and is guided to the docking imaging camera 42.

The cutting imaging channel includes a cutting imaging camera 46, preferably an infrared video camera, and appropriate cutting imaging optics 48 such as focusing lenses and reflecting mirrors. The cutting imaging optics 48 are mounted on the objective 27 and move with the objective 27. The cutting imaging camera 46 may be fixedly mounted to the laser system and remain stationary, or it may be mounted on the cutting imaging optics 48 and move with the objective 27 and optics 48. When the cutting imaging channel is operated during laser treatment, the objective 27 is located at an operation position which is directly above the laser beam exit 31 so that the laser beam from the objective is able to pass through the beam exit throughout the XY scanning. At this position, the cutting imaging optics 48 are optically aligned with the cutting imaging camera 46 (either stationary or moving with the optics 48) to direct reflected light from the eye to the cutting imaging camera 46. Thus, if the cutting imaging camera 46 is stationary, it is located at a position which optically aligns with the cutting imaging optics 48 when the latter is at the operation position. At the operation position, the objective 27 blocks the light paths of the visual field of the docking imaging channel.

In the illustrated embodiment, a mirror TM1 reflects the laser beam from the scanline generation module traveling in the X direction to the −Y direction toward a beam splitter TM2 (part of the cutting imaging optics 48), which in turn reflects the laser beam from the −Y direction to the Z direction into the objective 27 and toward the eye. The light reflected from the eye is collected by the objective 27 and passes through the beam splitter TM2, and is further guided by the cutting imaging optics 48 into the cutting imaging camera 46.

It should be noted that FIG. 1 is a 2-dimensional depiction of a 3-dimensional spatial arrangement and the mirrors TM1 and TM2 are not depicted in their actual spatial orientation. In FIG. 1, the coordinates indicated in the lower-right hand corner represent the coordinates relevant to the objective 27, the XY stage 28, and the docking and cutting imaging channels.

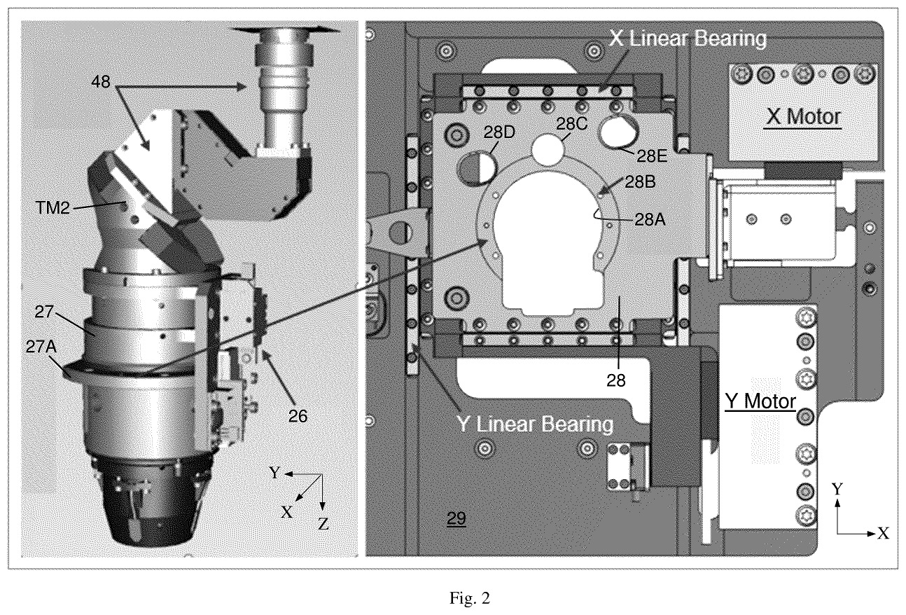

The spatial arrangements of the objective 27, the XY stage 28, and the docking and cutting imaging channels are illustrated in further detail in FIGS. 2 and 3. The left hand side of FIG. 2 shows a perspective view of the cutting objective 27 and the cutting imaging optics 48 (enclosed in a housing) affixed to the objective 27. As mentioned earlier, the cutting imaging camera 46 (not shown in FIG. 2) is either fixedly mounted on the laser system and does not move with the cutting imaging optics 48, or mounted on the cutting imaging optics 48 and moves with it.

The right hand side of FIG. 2 shows a bottom plan view of the XY stage 28 and portions of the support frame 29 of the laser system. The XY stage 28 is coupled to the support frame 29 via an X linear bearing and a Y linear bearing, and is driven by an X motor and a Y motor to move along the bearings in X and Y directions relative to the support frame. The XY stage 28 defines a center opening 28A, with a mounting flange 28B around the opening; the objective 27 passes through the center opening and is mounted to the XY stage 28 via the mounting flange 28B and a corresponding circular flange 27A around the housing of the objective. This way, the moving XY stage 28 carries the objective 27 and moves it during cutting. A slow-Z motor 26 drives a lens combination within the objective 27 to move in the Z direction relative to the objective housing to accomplish slow-Z scanning (see also FIG. 1).

The XY stage 28 is also operable to move the cutting objective 27 (along with the cutting imaging optics 48, and the cutting imaging camera 46 if it is mounted on the cutting imaging optics) to the parking position when the docking imaging channel is operated during docking and undocking. FIG. 3 (bottom perspective view) shows the cutting objective 27 at the parking position, which in this example is off in the −Y direction relative to the laser beam exit (cone optics 31). Note that the objective 27 and XY stage 28 are enclosed within a housing of the laser system, not shown in FIG. 3, which also supports the cone optics 31 in a stationary position.

As shown in FIGS. 2 and 3, the XY stage 28 further defines three smaller openings 28C, 28D and 28E. The first small opening 28C is aligned with the center opening 28A in the Y direction; the other two openings 28D and 28E are located on two sides of and spaced apart from the opening 28C in the X direction (and also in the Y direction in this example). When the objective 27 is moved to the parking position, the first small opening 28C is aligned with the beam exit (cone optics 31), forming a docking imaging path between the beam exit and the docking imaging optics 44 to allow the reflected light from the eye to enter the docking imaging channel, as well as to allow the fixation light to reach the eye. The other two small openings 28D and 28E are located to form two docking illumination paths to allow two docking illumination light beams, generated by two respective docking illumination light sources (not shown in the drawings), to illuminate the eye during docking. Note that although FIG. 3 depicts the docking image path and the docking illumination paths as “tubes”, this is only for purposes of explanation; there is in fact no physical structure located between the three small openings 28C-E and the cone optics 31 when the objective is in the parking position.

In alternative embodiments, the first small opening 28C, or all three small openings 28C-E, may be contiguous with the center opening 28A, so long as they allow the docking imaging path and the docking illumination paths to be formed when the cutting objective is moved to the parking position. In other alternative embodiments, the XY stage may be moved entirely out of the light paths of the docking imaging channel. In further alternative embodiment, in lieu of the two small openings 28D and 28E, the two docking illumination light sources may be directly mounted on the XY stage 28 to illuminate the eye.

An advantage of the dual channel imaging system according to embodiments of the present invention is that, for the cutting imaging channel, a small numerical aperture can be used so that the cutting image has sufficient depth of focus and can clearly image both photodisruption bubble image at the plane of laser treatment (e.g., incision) and the image of the pupil which is typically 2-4 mm below the patient interface.

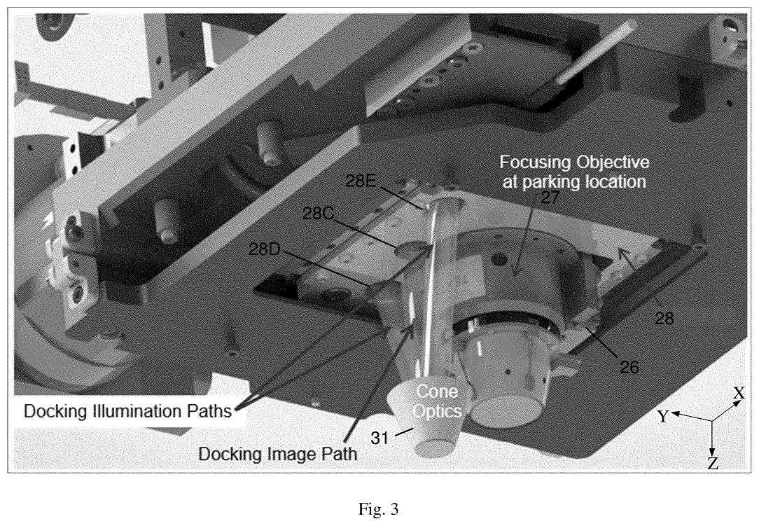

During laser treatment, images captured by the docking imaging channel and the cutting imaging channel may be displayed on the display device 18. FIG. 4 illustrates a composite image formed by a stationary image of the docked eye captured by the docking imaging channel immediately before the start of the laser scan (referred to as the docking image) with images captured by the cutting imaging channel during treatment (referred to as the cutting images) overlayed on it (i.e. replacing the corresponding portion of the docking image). Each overlayed cutting image is located at the actual location of the cutting FoV relative to the docking FoV and has the same object-to-image ratio as the docking image. The boundary of the current cutting FoV, which moves across the stationary docking image, is indicated by a circle, and the laser scanline is located along a diameter of the current cutting FoV. In this example, the FoV of the docking image is approximately 13 mm in diameter, and the FoV of the cutting image is approximately 2 mm in diameter. In the example shown in FIG. 4, various circles and curves representing the planned laser treatment pattern being executed are also superimposed on the docking and cutting images.

As mentioned earlier, the cutting imaging camera 46 may be mounted on and moving with the cutting imaging optics 48, in which case the cutting FoV formed on the cutting imaging camera 46 is stationary on the camera. In such a case, the positions of the cutting FoV relative to the stationary docking image, for purpose of the composite display, may be calculated from the scanning position of the XY stage 28. In the alternative embodiment where the cutting imaging camera 46 is mounted on and remains stationary relative to the laser system and does not move with the objective 27, the cutting FoV formed by the cutting imaging optics 48 moves on the cutting imaging camera 46. In such a case, the cutting images can be cropped from the larger image area of the cutting imaging camera 46 and overlayed on the stationary docking image at positions corresponding to their positions on the cutting imaging camera 46, provided that the frame of the cutting imaging camera 46 and the frame of the docking image camera have been properly aligned and scaled to a 1:1 ratio. The 1:1 imaging ratio is advantageous because the moving image can be directly stitched to the stationary background image.

To form the composite image during laser treatment, portions of the docking image are continuously displayed as a stationary image, while the live cutting image is continuously updated and moves as the cutting objective is scanned in the XY direction. Moreover, for portions of the docking image that has already been scanned by the cutting FoV, the docking image is replaced by the past cutting images at corresponding locations. In other words, during laser treatment, the displayed composite image are formed of three parts: First, for the portions that have not been scanned by the cutting FoV, the stationary docking image is displayed; second, for portions that have already been scanned by the cutting FoV, the stationary docking image is replaced by the past cutting images; and third, the live cutting image is displayed for the area that is currently under cutting.

In the example of FIG. 4, the photodisruption bubble pattern can be clearly seen (in this example, the cutting FoV is moving to the right and the photodisruption bubbles are present in the left half of the cutting FoV as well as the already scanned portion to the left of the current cutting FoV). This is helpful to the surgeon, as it is important for the surgeon to see the concurrent photodisruption bubble pattern.

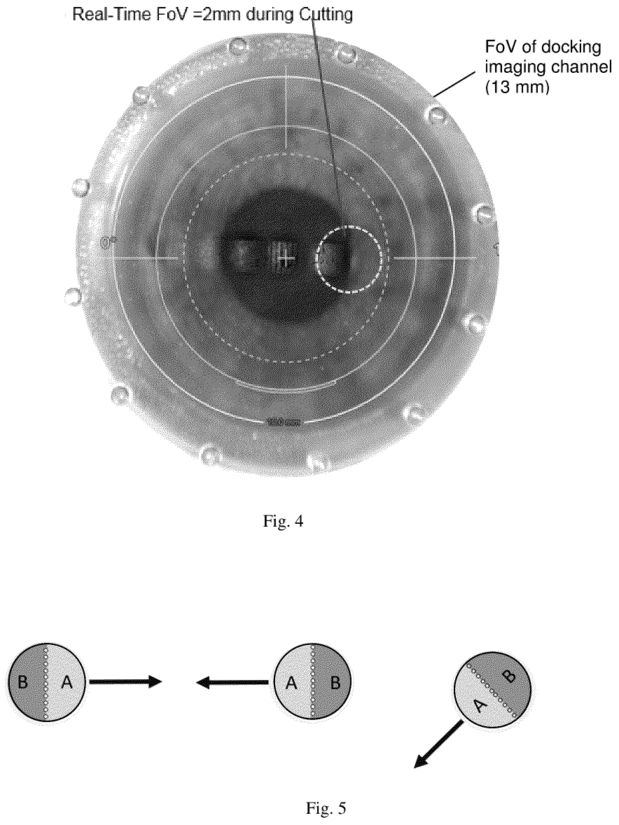

The images of the cutting imaging channel may be used during laser treatment for various monitoring and control purposes. FIG. 5 schematically illustrates three examples of the FoV of the cutting imaging channel during laser treatment. In each example, the circle represents the FoV boundary, the line of small circles represents the laser scanline, and the arrow outside of the FoV represents the XY scanning direction of the cutting objective, which is typically perpendicular to the scanline. The three examples in FIG. 5 show different cutting objective movement directions. As shown in FIG. 5, the circular FoV is divided by the laser scanline into two halves, namely, a front part A located in front of the scanline (i.e., the front half relative to the scanning direction, which includes areas not yet scanned by the laser pulses), and rear part B located behind the scanline (i.e. the rear half relative to the scanning direction, which includes areas just scanned by the laser pulses). As part A shows the tissue before laser treatment, this part of the image may be used to compare with the corresponding portion of the stationary docking image in real time to monitor eye movement in real time. Part B shows the real-time photodisruption bubble pattern after laser treatment.

Any suitable image comparison algorithms may be used to compare part A of the cutting image with the corresponding portion of the docking image. In one example, an edge detection algorithm is used to detect edges in both image parts, and the detected edge positions are compared. This is particularly effective when the cutting FoV crosses the pupil boundary. If the difference in the edge positions is greater than a predefined threshold, such as 200 μm, an alarm may be generated to alert the surgeon, or alternatively, the laser treatment process may be automatically paused or stopped.

In an alternative embodiment, for better eye movement detection during treatment, the stationary docking image shown on the display and used for image comparison is itself a composite image. For example, within a circular diameter of 7 mm, the composite docking image may come from an original docking image that was focused on the pupil and iris, and for the annulus with diameter between 7-12 mm, the composite docking image may come from another original docking image that was focused on the patient interface so that the cornea is clearly imaged. Both original docking images should be captured immediately before the start of laser treatment.

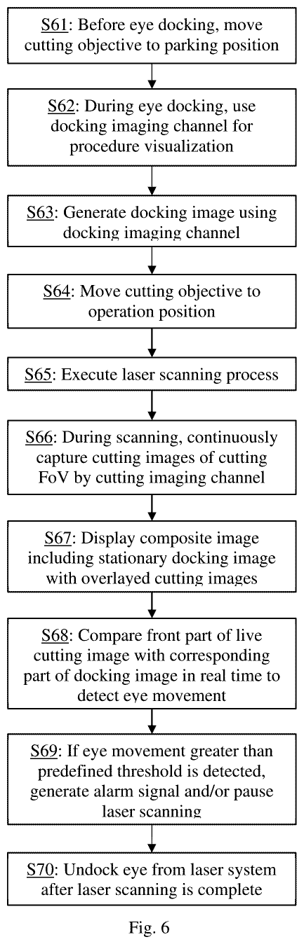

FIG. 6 schematically illustrates a method implemented in the laser eye surgery system according to an embodiment of the present invention. Unless otherwise stated, the steps are performed by the controller 12 controlling the various laser system components, either under the command of the surgeon or automatically based on a control program or both.

Before docking the eye to the laser system via the patient interface, the cutting objective 27 is moved to the parking position by the XY stage, out of the light paths of the visual field of the docking imaging channel (step S61). The eye is docked to the laser system via the patient interface, and in this process, the docking imaging is continuously captured by the docking imaging channel and displayed on the display device to aid the surgeon in procedure visualization (step S62). The docking is typically performed manually by the surgeon. After docking, a docking image is generated using the docking imaging channel (step S63). The docking image may be a composite image based on multiple original docking images. The cutting objective is then moved by the XY stage to the operation position where it is located directly above the laser beam exit (step S64).

The laser scanning process is then executed (step S65), during which the controller 12 controls the fast-Z scanner 25, the resonant scanner 21, the scanline rotator 23, the XY stage 28, and the slow-Z motor 26 to cooperate with each other to move the laser scanline on or within the eye tissue to perform treatment (e.g., form incisions). During the scanning process, the cutting imaging channel continuously captures images of the cutting FoV through the cutting objective (step S66).

A composite image is generated and displayed on the display device, which includes the docking image as a stationary image with the cutting images overlayed on it, including both the live cutting image and past cutting images that replace parts of the stationary docking image (step S67). The surgeon may monitor the scanning process by observing the photodisruption bubble pattern in the composite image. Meanwhile, the controller compares the front part of the live cutting image with the corresponding part of the docking image in real time to detect any eye movement (step S68). If eye movement greater than a predefined threshold is detected, the controller generates an alarm signal and/or automatically pauses laser beam delivery (step S69). After the scanning process is completed, the eye is undocked from the laser system (step S70).

It will be apparent to those skilled in the art that various modification and variations can be made in the laser eye surgery systems and methods of the present invention without departing from the spirit or scope of the invention. Thus, it is intended that the present invention cover modifications and variations that come within the scope of the appended claims and their equivalents.

1. A laser eye surgery system for treating a patient's eye, comprising:
a laser device configured to generate a pulsed laser beam;
an objective configured to focus the pulsed laser beam to a laser focus spot in the eye;
a movable stage, wherein the objective is mounted on the movable stage and the movable stage is configured to move the objective in two lateral directions perpendicular to an optical axis of the objective to scan the laser focus spot in the two lateral directions;
a first imaging channel including a first camera and a first set of optics associated with the first camera, wherein the first set of optics is configured to direct a reflected light from the eye into the first camera, and wherein the first imaging channel is configured to generate images of a first field of view (FoV) of the eye; and
a second imaging channel including a second camera and a second set of optics associated with the second camera, wherein the second set of optics is mounted on the objective and moves with the objective, wherein the second imaging channel is configured to generate images of a second FoV of the eye which is smaller than the first FoV;
wherein the movable stage is further configured to move the objective and the second set of optics between an operation position at which the laser beam passing through the objective is delivered through a beam exit of the laser surgery system to the eye and the reflected light from the eye is collected by the objective to enter the second set of optics, and a parking position at which the objective and the second set of optics are out of a light path between the beam exit and the first imaging channel.

2. The laser eye surgery system of claim 1, wherein the first FoV has a diameter of 10-13 mm and the second FoV has a diameter of 1 to 3 mm.

3. The laser eye surgery system of claim 1, wherein the first imaging channel is stationary relative to a frame of the laser eye surgery system and optically aligned with the beam exit.

4. The laser eye surgery system of claim 3, wherein the first set of optics includes a first beam splitter configured to direct the reflected light from the eye into the first camera, wherein the laser eye surgery system further comprises a fixation light source configured to generate a fixation light, and wherein the first beam splitter is configured to couple the fixation light into the eye.

5. The laser eye surgery system of claim 1, wherein the second camera is stationary relative to a frame of the laser eye surgery system, and optically aligned with the second set of optics when the objective and the second set of optics are moved to the operation position.

6. The laser eye surgery system of claim 1, wherein the second camera is mounted on and moves with the second set of optics.

7. The laser eye surgery system of claim 1, wherein the second set of optics includes a second beam splitter configured to direct the laser beam into the objective and to direct the reflected light from the eye into the second camera.

8. The laser eye surgery system of claim 1, wherein the movable stage defines a first opening and a second opening offset from the first opening, wherein the objective passes through the first opening and is mounted on the movable stage, wherein when the movable stage moves the objective to the parking position, the second opening is aligned with the beam exit to form a docking imaging path for the second imaging channel.

9. The laser eye surgery system of claim 1, further comprising at least one docking illumination light source configured to generate at least one docking illumination light beam, wherein the movable stage further defines at least one third opening, wherein when the movable stage moves the objective to the parking position, the at least one docking illumination light beam passes through the at least one third opening to illuminate the eye.

10. The laser eye surgery system of claim 1, further comprising a scanline generating module located upstream of the objective and configured to generate scanline of the pulsed laser beam, the scanline generating module including a lens movable in a laser beam propagation direction to scan the laser focus spot in the laser beam propagation direction, a resonant scanner configured to scan the laser beam in a plane perpendicular to the laser beam propagation direction to form the scanline, and a scanline rotator configured to rotate the scanline around the laser beam propagation direction.

11. The laser eye surgery system of claim 1, further comprising:
a display device; and
a controller operatively coupled to the laser device, the movable stage, the first imaging channel, the second imaging channel, and the display device, wherein the controller is configured to execute a control program to:
operate the laser device and the movable stage to scan the laser focus spot in the eye to perform a laser treatment;
operate the first imaging channel to generate a first image of the eye before performing the laser treatment, the first image covering the first FoV; and
while performing the laser treatment:
operate the second imaging channel to generate a plurality of second images of the eye, the second images covering the second FoV which moves across the eye;
generate a composite image of the eye, including the first image as a stationary image and the plurality of second images overlayed on the first image to replace corresponding portions of the first image, wherein each overlayed second image is located at a corresponding location defined by the second FoV relative to the first FoV and has a same object-to-image ratio as the first image, the composite image further including an indication of a current boundary of the second FoV; and
display the composite image on the display device.

12. The laser eye surgery system of claim 11, wherein the controller is further configured to execute the control program to, while performing the laser treatment, compare the plurality of second images to the first image in real time to detect any eye movement, including, for each second image, comparing a portion of the second image which has not been scanned by the laser focus spot to a corresponding portion of the first image to detect any eye movement.

13. The laser eye surgery system of claim 11, wherein the first image of the eye is a composite image generated from two or more original images of the eye captured by the first imaging channel before performing the laser treatment, wherein the two or more original images are focused on different structures of the eye.

14. The laser eye surgery system of claim 1, further comprising a controller operatively coupled to the laser device, the movable stage, the first imaging channel, and the second imaging channel, wherein the controller is configured to execute a control program to:
operate the laser device and the movable stage to scan the laser focus spot in the eye to perform a laser treatment;
operate the first imaging channel to generate a first image of the eye before performing the laser treatment, the first image covering the first FoV; and
while performing the laser treatment:
operate the second imaging channel to generate a plurality of second images of the eye, the second images covering the second FoV which moves across the eye; and
compare the plurality of second images to the first image in real time to detect any eye movement, including, for each second image, comparing a portion of the second image which has not been scanned by the laser focus spot to a corresponding portion of the first image to detect any eye movement.

15. The laser eye surgery system of claim 14, wherein the first image of the eye is a composite image generated from two or more original images of the eye captured using the first imaging channel before performing the laser treatment, wherein the two or more original images are focused on different structures of the eye.

16. A method implemented in a laser eye surgery system to treat a patient's eye, the system comprising a laser device configured to generate a pulsed laser beam, an objective configured to focus the pulsed laser beam to a laser focus spot in the eye, a movable stage configured to move the objective in two lateral directions perpendicular to an optical axis of the objective, a first imaging channel configured to generate images of a first field of view (FoV) of the eye, a second imaging channel configured to generate images of a second FoV of the eye which is smaller than the first FoV, and a display device, the method comprising:
moving the objective to a parking position which is out of a light paths of a visual field of the first imaging channel;
while the eye is being docked to the laser system, continuously capturing first images of the eye by the first imaging channel and displaying the first images on the display device;
after the eye is docked to the laser system, generating a first stationary image of the eye by the first imaging channel, the first image covering the first FoV;
moving the objective to an operation position at which the laser beam passing through the objective is delivered to the eye;
scanning the objective to deliver a laser focus spot in the eye to perform a laser treatment;
while performing the laser treatment:
collecting and directing reflected light from the eye through the objective to the second imaging channel;
continuously capturing a plurality of second images of the eye by the second imaging channel, the plurality of second images covering the second FoV which moves across the eye;
generating a composite image of the eye, including the first stationary image as a stationary image and the plurality of second images overlayed on the first stationary image to replace corresponding portions of the first stationary image, wherein each overlayed second image is located at a corresponding location defined by the second FoV relative to the first FoV and has a same object-to-image ratio as the first image, the composite image further including an indication of a current boundary of the second FoV;
displaying the composite image on the display device;
comparing the plurality of second images to the first stationary image in real time to detect any eye movement, including, for each second image, comparing a portion of the second image which has not been scanned by the laser focus spot to a corresponding portion of the first stationary image to detect any eye movement; and
generating an alarm signal or automatically pausing laser beam delivery when detecting an eye movement greater than a predefined threshold; and
after performing the laser treatment, undocking the eye from the laser system.

17. The method of claim 16, wherein the first FoV has a diameter of 10-13 mm and the second FoV has a diameter of 1 to 3 mm.

18. The method of claim 16, wherein the steps of moving the objective to a parking position, moving the objective to an operation, and scanning the objective position are performed by the movable stage.

19. The method of claim 16, further comprising:
while the eye is being docked to the laser system, delivering a fixation light and an illumination light to the eye.

20. The method of claim 16, wherein the step of generating the first stationary image of the eye includes:
capturing two or more original images of the eye using the first imaging channel before performing the laser treatment, wherein the two or more original images are focused on different structures of the eye; and
generating the first stationary image as a composite image from the two or more original images.
